(19) United States
(12) Patent Application Publication
(10) Pub. No.: US 2004/0153031 A1
(43) Pub. Date: Aug. 5, 2004

(54) Injection guide for locating sites on a user's body

(76) Inventor: Johannes Cornelius Theodorus Van Kaauwen, Surrey (CA)

Correspondence Address: DANIEL B. SCHEIN, P. O. BOX 28403, SAN JOSE, CA, 95159, US

(21) Appl. No.: 10/469,179

(22) PCT Filed: Feb. 27, 2001
(86) PCT No.: PCT/CA01/00239

(51) Int. Cl. A61M 5/00

(52) U.S. Cl. 604/116

(57) ABSTRACT

An injection guide (1) for identifying injection sites on different areas of a user's body. The injection guide comprises a flexible sheet (5) having first and second surfaces, and a plurality of holes (10) passing therethrough. The holes (10) are associated with number indicia (12) to indicate the days of the month. Some of the holes (10) are associated with more than one number indicia (12) and each number indicia is appropriately oriented to be read from the user's perspective when in use on the different areas of the body. Paths (15, 16) of safe injection sites for the different areas of the user's body are defined by path indicia that extend between the holes (10). A positioning system on the injection guide assists the user in consistently positioning the injection guide on each different area of the user's body. When using the injection guide, the user may mark the injection site at the appropriate hole, corresponding to the day of the month, or the user may administer the injection directly thought the appropriate hole (10).

### FIELD OF THE INVENTION

The present invention relates generally to an injection guide, and more particularly, to an injection guide for systematically rotating injection sites over different areas of a user's body to avoid repeated injections at the same location.

### BACKGROUND OF THE INVENTION

Diabetes is a disease that occurs when the body cannot make use of glucose in the blood for energy because either the pancreas is not able to make enough insulin or the insulin that is available is not effective. In many cases, the disease can be controlled with oral medication, diet, and exercise. In other cases, however, the disease can only be controlled by regular insulin subcutaneous injections.

Insulin injections must be received daily and in certain cases must be received several times a day. The difficulty is that only certain parts of the body may be safely used for such injections without excessive discomfort or risk of penetrating major veins, arteries or muscle areas. Moreover, tissue damage caused by an injection generally requires four to six weeks to heal. Repeated injection at the same site before the site is allowed to heal may result in tissue damage that causes impaired absorption of insulin or other injectable medicines.

To ensure an equal absorption and distribution of insulin or other injectable medicines and to avoid excessive tissue damage, a system for identifying and rotating injection sites is needed. Previous equipment for accomplishing this task are disclosed in the following patents:

U.S. Pat. No. 3,542,022 to Bartnik

U.S. Pat. No. 4,228,796 to Gardiner

U.S. Pat. No. 4,362,157 to Keeth, and

Canadian Patent No. 1,140,016 to Berger

Canadian Patent No. 1,140,016 and U.S. Pat. No. 4,228,796 disclose injection guides that are strapped onto the leg of the user and are thereby limited to injection sites on the thigh. U.S. Pat. No. 3,542,022 discloses a template guide for defining a general region on the buttocks for injecting medication intramuscularly. U.S. Pat. No. 4,362,157 to Keeth discloses a template that relies on dye released by puncturing of the template by a needle to identify previously used injection sites.

There are three ways of injecting a liquid into the body: intravenously, intramuscularly and subcutaneously. There is a need for a device that enables users to consistently and systematically identify safe subcutaneous injection sites on different areas of the body whether the user is assisting a patient or the user is self-administering their own injections.

### SUMMARY OF THE INVENTION

The present invention provides an injection guide that permits easy separation of subcutaneous injection sites in a given body area. While the following discussion concentrates on use of the guide to inject insulin, it will be appreciated that the guide is not limited to use by diabetics. Subcutaneous injections at regular intervals are required by some patients with multiple sclerosis and patients with Hepatitis C. Similarly, some types of cancer are treated by drugs administered regularly by subcutaneous injection. All such injections can be administered using the guide of the present invention.

According to a broad aspect, the invention provides an injection guide for identifying subcutaneous injection sites on different areas of a user's body. The injection guide is comprised of a flexible sheet having first and second surfaces, and a plurality of holes passing therethrough. The holes are associated with number indicia to indicate the days of the month. Some of the holes are associated with more than one number indicia and each number indicia is appropriately oriented to be read from the user's perspective when in use on the different areas of the body. Paths of safe injection sites for the different areas of the user's body are defined by path indicia that extend between the holes. A positioning system on the injection guide assists the user in consistently positioning the injection guide on each different area of the user's body. When using the injection guide, the user may mark the injection site at the appropriate hole, corresponding to the day of the month, or the user may administer the injection directly through the appropriate hole.

Other aspects and features of the present invention will become apparent to those ordinarily skilled in the art upon review of the following description of specific embodiments to the invention in conjunction with the accompanying drawings.

### BRIEF DESCRIPTION OF THE DRAWINGS

Embodiments of the invention will now be described with reference to the attached drawings wherein:

FIGS. 1a and 1b are plan views of a first surface of an injection guide made according to preferred embodiments of the invention;

FIGS. 2a and 2b are plan views showing the second surface of the injection guide of FIGS. 1a and 1b, respectively; and

FIGS. 3a and 3b are front and back views, respectively, showing areas of the human body where the injection guide according to this invention may be used.

It will be appreciated that for simplicity and clarity of illustration, elements illustrated in the accompanying drawings have not necessarily been drawn to scale. Further, where considered appropriate, reference numerals and labels have been repeated among the drawings to indicate corresponding or analogous elements.

### DETAILED DESCRIPTION

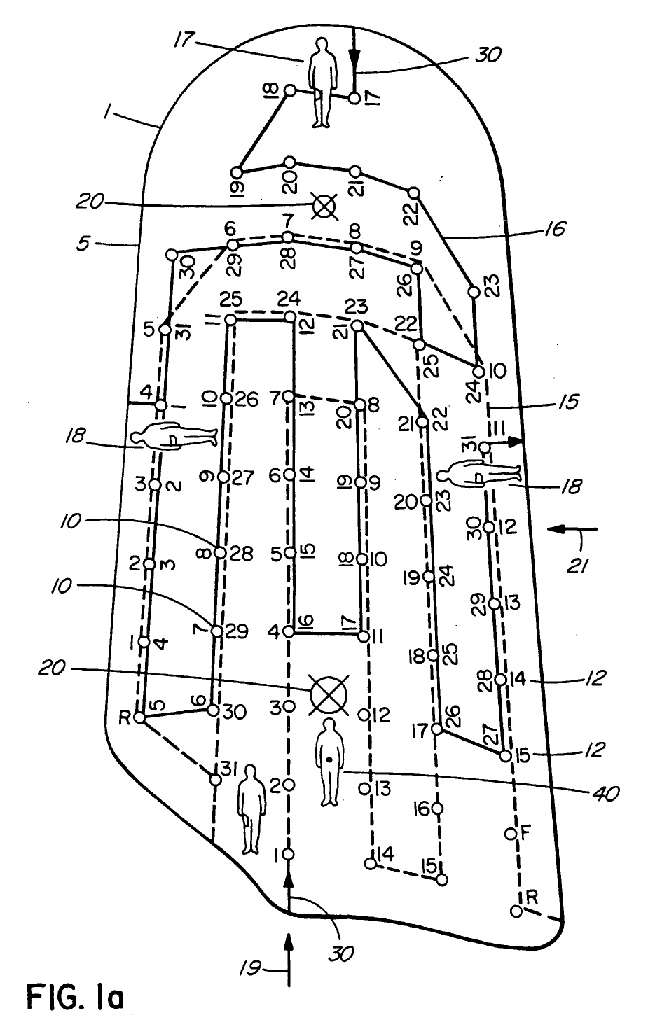

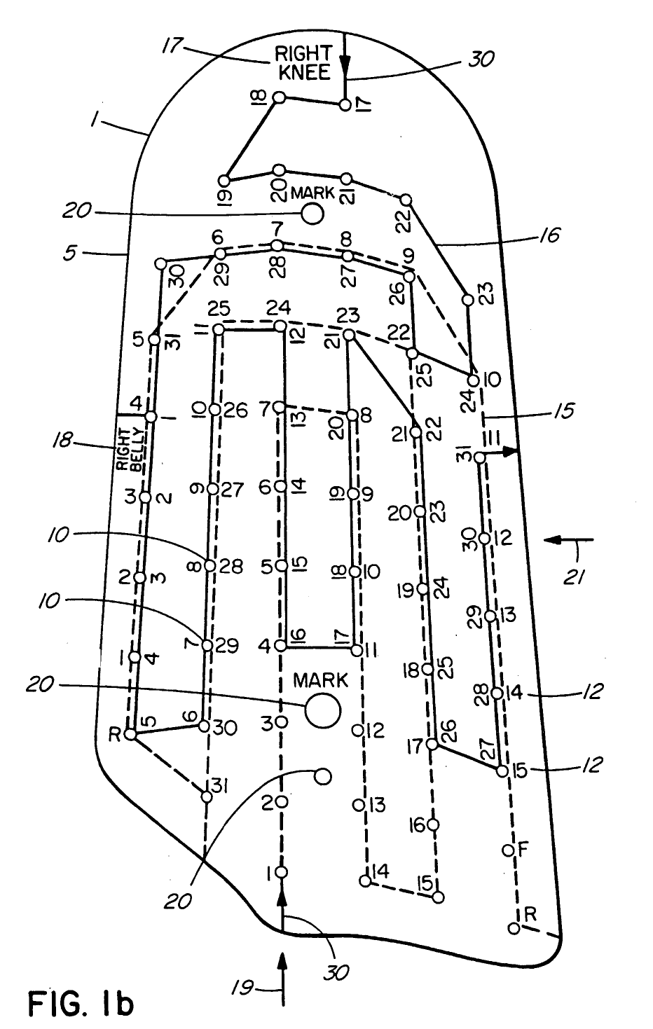

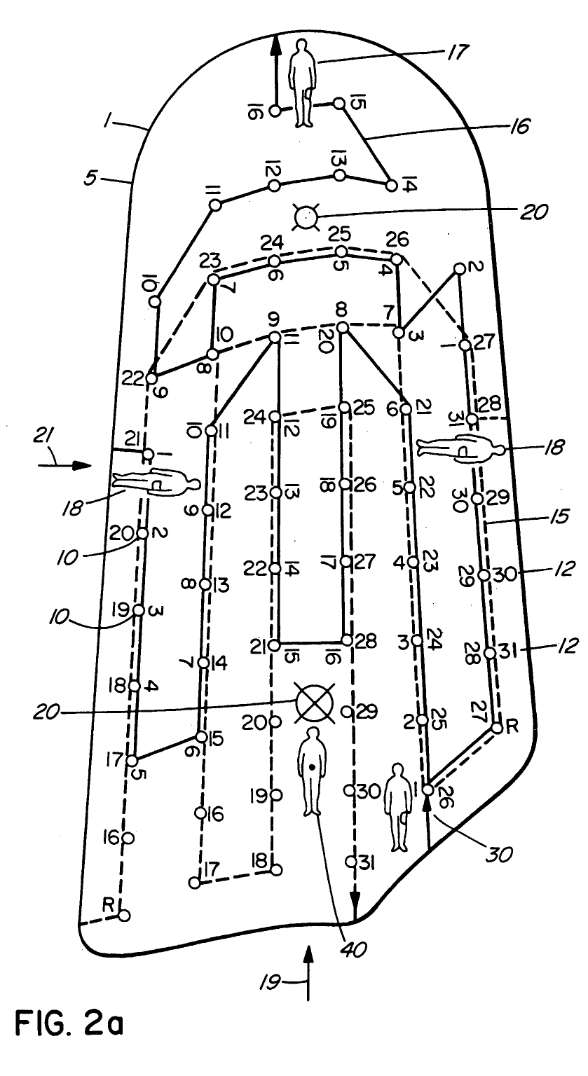

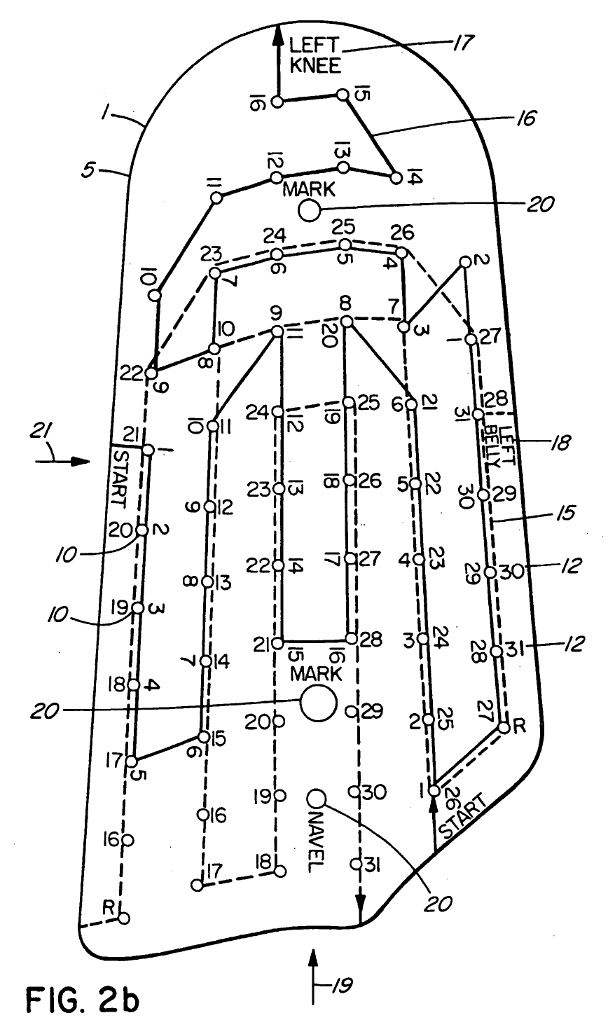

Referring to FIGS. 1a, 1b and 2a, 2b there is shown an injection guide 1 according to preferred embodiments of the present invention comprising a flexible sheet 5 of material adapted to be draped over an area of the body to locate injection sites for a hypodermic needle. The guide is primarily designed for use by diabetics to inject insulin, however, it will be apparent to those skilled in the art that the injection guide of the present invention can be used with any medication that requires multiple injections sub-cutaneously via a needle. Injections can be self-administered or performed by an assistant. In the following description, reference to a user injecting themselves also covers an assistant injecting a patient and vice versa.

Flexible sheet 5 is preferably formed from nylon, vinyl, rubber, soft plastic, or the like so that the sheet will readily conform to the body part over which it is draped. Such materials also permit the sheet to be folded or rolled compactly for convenient storage in between uses.

Flexible sheet 5 has first and second surfaces and a plurality of holes 10 passing therethrough. The plurality of holes 10 are associated with number indicia 12 to indicate days of the month to assist the user in selecting the proper hole for administering an injection. At least some of the holes 10 are associated with more than one number indicia 12 wherein each number indicia 12 is appropriately oriented to be read from the user's perspective when in use on different areas of the body. For example, the guide can be positioned on the user's left or right thigh or on the user's left or right abdomen for self-administered injections. Depending on the area to be injected, the appropriate surface of the guide is selected by the user and placed on the desired body area. To assist the user in properly orienting the injection guide, labels 17, 18 can be provided. For example, the surface illustrated in FIGS. 1a and 1b is intended to be positioned on the right thigh or the right abdomen of a user. When in use on the right thigh, label 17 in FIGS. 1a and 1b should be positioned adjacent the right knee. In one embodiment, the label 17 may read “RIGHT KNEE” as shown in FIG. 1b. In another embodiment, as shown in FIG. 1a, the label is a universally understood symbol depicting the injection guide on the right thigh of a human form. All the number indicia 12 associated with injection sites for the right thigh are oriented to be read from the user's perspective. In FIGS. 1a and 1b, the guide would be viewed by a user from the point of view of arrow 19 when on the right thigh, and there is a set of number indicia 12 oriented to be read from this perspective. Similarly, when in use on the right abdomen, label 18 is positioned adjacent the right side of the belly. In one embodiment, the label may read “RIGHT BELLY” as shown in FIG. 1b. In another embodiment, as shown in FIG. 1a, the label is a universally understood symbol depicting the injection guide on the right abdomen of a human form. All number indicia 12 associated with injection sites for the right belly or abdomen are oriented to be read from the user's point of view indicated by arrow 21.

Furthermore, it is preferable that path indicia 15, 16 extend between holes 10 to define paths of safe injection sites for different areas of a user's body. In other words, path indicia in the form of printed lines 15, 16 connect together holes 10 into groups that identify a series of safe injection sites. In FIG. 1, path 15 indicated by dashed lines is used to join holes that define injection sites on the thigh. Path 16 is used to interconnect holes that define safe injection sites on the abdomen. Preferably, each printed line, the associated number indicia, and the label to assist in orientation are all printed in the same colour to aid the user in distinguishing the various sets of markings. For example, the path indicia adapted for use on the user's thigh 15 may be blue and the path indicia adapted for use on the user's abdomen 16 may be red. In this way, the user is able to easily determine which path should be followed to identify safe injection sites for that particular area of the body. Where there is more than one path 15, 16 indicated on the flexible sheet 5, a hole 10 may be included in both paths 15, 16 and, therefore, may be associated with more than one number indicia 12. To clearly distinguish the number indicia 12, each number indicia 12 is in a colour and orientation corresponding to the particular path 15, 16 to which it relates. In this way, the user is able to easily distinguish the paths 15, 16 and identify the appropriate hole for administering the injection. In addition, arrow heads 30 are included in paths 15, 16 to assist the user in finding the starting point of a particular path 15, 16, in following the path in the appropriate direction and to direct the user between the surfaces of the guide.

FIGS. 2a and 2b illustrate the second surface of the injection guide in which labels 17 and 18 assist in the proper orientation of the surface on the left side of the body on the thigh or abdomen. In one embodiment, as shown in FIG. 2b, the labels 17 and 18 read “LEFT KNEE” and “LEFT BELLY”, respectively, and the label “START” is preferably printed adjacent the holes labeled to correspond to the first day of a month to assist the user in locating the first hole. In another embodiment, as shown in FIG. 2a, the labels 17 and 18 are universally understood symbols depicting the injection guide on the left thigh and left abdomen of a human form, respectively.

A positioning system is also provided on injection guide 1 to assist the user in consistently positioning the injection guide on each different area of the user's body. Preferably, the positioning system is in the form of at least one hole 20 alignable with an artificial mark or natural landmark on the user's body. Preferably, holes 20 are labelled to assist the user in their proper use depending on the area of the body being injected. For example, in one embodiment, as shown in FIGS. 1b and 2b, one of positioning holes 20 is marked with the label “NAVEL” to indicate that this hole is aligned with the natural landmark of the user's navel to properly orient the guide on the abdomen. In another embodiment, shown in FIGS. 1a and 2a, positioning hole 20, that is to be aligned with the user's navel, is labeled with a universally understood symbol 40 that depicts a human form with a mark corresponding to the navel. It will be noted that the symbol is properly oriented to be readable by the user when in place on the abdomen. Temporary ink marks, preferably with semi-permanent ink, are made on the skin of the thigh to align with the holes for consistent positioning.

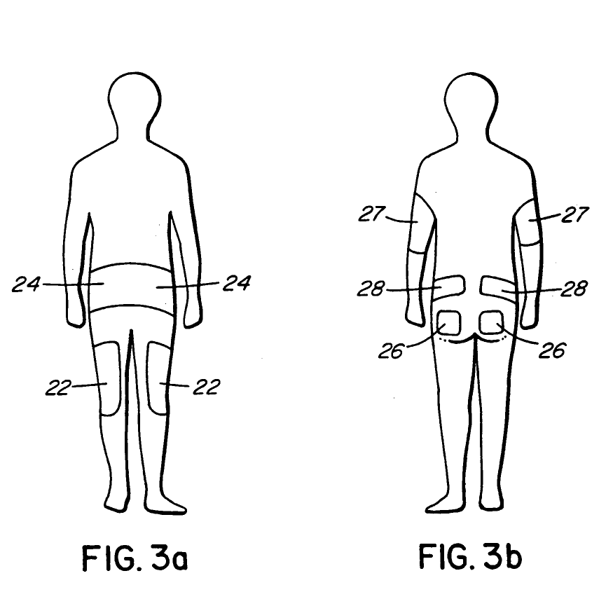

In use, the injection guide 1 is placed with the flexible sheet 5 over the chosen area of the user's body. The areas for self-administered injections are the upper areas 22 of each thigh and the left and right sides of the abdomen 24 as best shown in FIG. 3a. The injection guide of the present invention can also be used on other areas of the body including the buttocks 26, the side and rear of each upper arm 27 and the lower back 28 as shown in FIG. 3b. Injection into the sites of FIG. 3b require assistance by another person.

Once the flexible sheet 5 is positioned on the chosen area of the user's body, the appropriate hole 10 to administer an injection is selected based on the date. To select the appropriate hole 10 for injection, the user simply follows the defined path 15, 16 of safe injection sites for the particular area of the body chosen, and locates the hole 10 corresponding to that particular day of the month. In this way, the only thing the user has to remember is the day of the month to be able to choose a safe site for injection. Preferably, for areas where injections can be self-administered, the user places a temporary mark, for example with a felt-pen, through the selected hole 10 and removes the flexible sheet 5 from the selected area of the body. The user may then administer the injection about one centimeter within the proximity of the temporary mark. Alternatively, the user may administer the injection directly through the selected hole 10. When another injection is required, the user may position the flexible sheet 5 in substantially the same position as before by aligning at least one of the positioning system holes 20 with an artificial mark, such as a pen mark, or a natural landmark, such as the navel, depending on the area of the body chosen. Preferably, the artificial mark is made with a semi-permanent ink so that the mark may be re-used to position the flexible sheet 5 on the chosen area of the user's body.

As described above, both surfaces of flexible sheet 5 are used. The first surface of the flexible sheet 5 may be adapted for use at areas on one of the left or the right sides of the user's body; and the second surface may be adapted for use at areas on the other side of the user's body. The user begins administering injections following one path 15 or 16 on the first surface of the sheet 5 adapted for use on one side of the body. Once the user has completed the path 15 or 16 on the first surface of the sheet 5, the user may continue administering injections in the same area but on the other side of the body by following the same path 15 or 16 on the second surface of the sheet 5. This process may be continued for different areas of the body. In this way, the number of injection sites available to the user may be multiplied. For example, an injection guide 1 might have a sufficient number of holes 10 arranged so that each full path 15, 16 has number indicia 12 corresponding to the days spanning the course of a month and a half (6 weeks). If one injection is administered per day, and the user utilizes only one area of the body by following one path 15 or 16, it may be possible to obtain a maximum of ninety-three injection sites with a turnaround time of three months, i.e., the tissue will have three months to recover. If the user also utilizes a second area of the body by interactively using both paths 15 and 16, a turnaround time of six months may be achieved with one injection per day.

It is also contemplated that the present invention may be used where the user must make multiple injections per day. For example, the user may inject up to four times daily with a turnaround time of at least 6 weeks by positioning the guide for each injection on one of the four available surfaces for self-injection (the left and right thigh, the left and right abdomen) and using the appropriately numbered hole corresponding to the day of the month. It will be readily apparent that if other areas of the body are also used, as shown in FIG. 3, the number of available injection sites is further multiplied, however, use of sites other than the thigh or abdomen require an assistant. If the user has an assistant, five body areas are available for injection to provide up to a total of 403 injection sites before the same site is used again. If a single injection is required per day, this translates into 403 days (13 months) before the user must return to the same injection site.

The guide of the present is adapted for use on bodies of various dimensions. For example, the guide may be safely used on a baby, child, or adult by limiting the useable area of the injection guide 1 to the portion of the paths 15, 16 that fits over the chosen body part. Preferably, paths 15, 16 include spaced, parallel sections of increasingly greater width to define paths of safe injection sites at body areas of increasing dimensions. Preferably, when used on a baby, use is limited to the central parallel section of paths 15, 16 such that each path 15, 16 has number indicia 12 corresponding to the days spanning the course of two weeks. Similarly, when used on a child, having larger body dimensions than a baby, an additional parallel section of paths 15, 16 is used such that each path 15, 16 has number indicia 12 corresponding to the days spanning the course of four weeks. An adult would be able to use all parallel sections of paths 15, 16 such that each path has number indicia 12 corresponding to the days spanning the course of six weeks.

While this invention has been described with reference to illustrative embodiments, this description is not intended to be construed in a limiting sense. Various modifications of the illustrative embodiments, as well as other embodiments of the invention, will be apparent to persons skilled in the art upon reference to this description. It is therefore contemplated that the appended claims will cover any such modifications and embodiments as fall within the true scope of the invention.

I claim:

1. An injection guide for identifying subcutaneous injection sites on different areas of a user's body comprising:
a flexible sheet having first and second surfaces, the sheet having a plurality of holes therethrough;
number indicia associated with the plurality of holes to indicate days of the month, at least some of the holes being associated with more than one number indicia and each number indicia being appropriately oriented to be read from the user's perspective when in use on the different areas of the body;
path indicia extending between holes to define paths of safe injection sites for the different areas of the user's body; and
a positioning system for consistently positioning the injection guide on each different area of the user's body.

2. An injection guide as claimed in claim 1 wherein the different areas of the user's body include the thighs, the abdomen, the buttocks, the lower back, and the upper arms.

3. An injection guide as claimed in claim 1 wherein the positioning system comprises at least one hole alignable with an artificial mark or natural landmark on the user's body.

4. An injection guide as claimed in claim 1 wherein the path indicia mark at least two paths of safe injection sites in which one path is adapted for use on the user's thigh and the second path is adapted for use on the user's abdomen.

5. An injection guide as claimed in claim 4 wherein the path indicia for each path of safe injection sites is a different colour.

6. An injection guide as claimed in claim 4 wherein the number indicia corresponding to each path of safe injection sites is a different colour.

7. An injection guide as claimed in claim 1 wherein the first surface of the flexible sheet is adapted for use at areas on one of the left or the right sides of the user's body; and the second surface is adapted for use at areas on the other side of the user's body.

8. An injection guide as claimed in claim 7 wherein said plurality of holes comprises a sufficient number of holes to indicate days in a month and a half.

9. An injection guide as claimed in claim 7 wherein the path indicia define paths that begin on the first surface and continue on the second surface of the flexible sheet.

10. An injection guide as claimed in claim 1 in which the flexible sheet is rubber.

11. An injection guide as claimed in claim 1 in which the flexible sheet is soft plastic.

12. An injection guide as claimed in claim 1 in which the flexible sheet is vinyl.

13. An injection guide as claimed in claim 1 in which the flexible sheet is nylon.

14. An injection guide as claimed in claim 1 in which the path indicia mark at least two paths of safe injection sites, each path including spaced, parallel sections to define paths of safe injection sites at body areas of different dimensions.

15. An injection guide as claimed in claim 14 in which the spaced, parallel sections are spaced to define paths of safe injection sites on the body areas of a baby, child or adult.
